(19) United States
(12) Patent Application Publication
(10) Pub. No.: US 2023/0197235 A1
(43) Pub. Date: Jun. 22, 2023

(54) Electronic Prescription System

(71) Applicant: Michael Lion, Annapolis, MD (US)

(72) Inventor: Michael Lion, Annapolis, MD (US)

(21) Appl. No.: 17/660,573

(22) Filed: Apr. 25, 2022

Related U.S. Application Data
(60) Provisional application No. 63/179,702, filed on Apr. 26, 2021.

(51) Int. Cl. G16H 20/13; G16H 10/60; G16H 40/67

(52) U.S. Cl. CPC G16H 20/13; G16H 10/60; G16H 40/67

(57) ABSTRACT

A combined electronic prescription and pharmacy adjudication software system. A computer, receives information from a patient indicative of the patient's identity, and receives information from a medical professional, indicative of a type of medication to be dispensed to the patient. The computer stores a hierarchy of different medications which are most likely to be approved by an insurance represented by the insurance information and quantities of the medications which are likely to be approved, and uses that to determine a medication to be dispensed.

This application claims priority from provisional application No. 63/179,702, filed Apr. 26, 2021, the entire contents of which are herewith incorporated by reference.

### BACKGROUND

Currently, doctors use electronic prescription systems such as EMR's to submit electronic prescriptions (“e-prescriptions) either to pharmacies directly or to other pharmacy software. The pharmacies accept the e-prescription, which is then processed by a pharmacy technician.

When a prescription is input into a pharmacy software system, it is submitted for approval to an insurer or pharmacy benefit manager (“PBM”). The insurer/PBM may approve or deny the claim. If the claim is not approved, then the pharmacy tech needs to either contact the doctor and get a new prescription for a different medication, or to change the quantity and resubmit. There can also be a long list of other manual changes in the pharmacy system to get the claim submitted correctly based on the rejection response from the insurer/PBM.

### SUMMARY

The inventor recognizes that this requires two different IT systems: the EMR and the pharmacy claim submitting software, which are physically separated, and hence do not share information.

The inventor recognizes that there is not a mechanism for automated mechanism for changing a insurer/PBM declined prescription.

This also means that when a paper prescription or the e-prescription is sent to a pharmacy software system, the pharmacy tech needs to add in the patient's specific information either manually, or by searching for the patient's PBM information.

An aspect of the system automatically determines how to change aspects of a prescription when the prescription is rejected.

Another aspect of the system checks coupon codes to ensure that technicians are not submitting coupons for improper claims, e.g., federal claims.

The system has described herein has new features including a closed system of e-Rx prescribing with an automatic electronic standing order, a driver license scanner which has a PBM look up, PBM claim filters and automatic PBM reject code responses.

Other aspects are described in the following.

### BRIEF DESCRIPTION OF THE DRAWINGS

The drawings show aspects of the invention, and specifically:

FIG. 1 shows the operation of checking the patient information;

FIG. 2 shows the operation of checking PBM information;

FIG. 3 shows the operation of the PBM claim;

FIG. 4 shows the operation of checking a standing order within a priority of settings;

FIG. 5 shows the e-prescription being sent for the pre-picked medication;

FIG. 6 shows picking the medication quantity;

FIG. 7 shows refill splits;

FIG. 8 shows the claim being submitted to the PBM and approved;

FIG. 9 shows allowing the user to choose a different medication;

FIG. 10 shows the medication being approved;

FIG. 11 shows the patient's co-pay being accepted;

FIG. 12 shows the patient signing for the medication; and

FIG. 13 shows the hardware system used by patient, pharmacy and prescriber.

### DETAILED DESCRIPTION

An embodiment describes a combined electronic prescription system combined with a pharmacy adjudication system, within the same or communicating IT system(s). This allows doctors and other prescription creators to easily send electronic prescriptions to the in house pharmacy.

The application describes a way of implementing an “electronic standing order”. Doctors and their offices normally have a lot of back and forth when sending an erx/rx to a retail pharmacy. The inventor recognized that this is because when the prescription doesn't get approved by the PBM (pharmacy benefit manager), then pharmacy needs to call the doctors office and have them change the erx/rx to a different erx/rx that would get covered. This process is continued until a prescription is agreed on and it covered.

The inventor recognizes that this is a very time consuming issue for both the doctor and the pharmacy.

Embodiments address that problem by having the physicians dispense and prescribe in the same platform/tech system at the point of care (rather than sending the erx/rx to a retail pharmacy). This can address “the back and forth” from the calls from a retail pharmacy. Also, because the doctors preset and pick according to a hierarchy, within the SAME platform (e-prescribing and pharmacy dispensing software combined) it allows the physician's office personnel to simply press a button to the next medication within that prepicked standing order, without the use of two systems (EMR and pharmacy software), and they can tell in seconds what medication is covered for that patient. That medication can be dispensed at the point of care, rather than having the patient going to a retail pharmacy or can be sent as conventional to a pharmacy.

The system allows pre-selecting of medications for certain classes of or types of medications. By pre-selecting these automatic predetermined electronic prescriptions, this decreases callbacks which will be otherwise caused by non-covered medications or medications needing prior authorization to doctor's offices from retail pharmacies. This also allows a doctor to quickly submit a new prescription to a patient, check PBM coverage, and select a medication that is most likely to be covered. This allows the patient to obtain access to the vital medications more quickly at the doctor's office, rather than at a retail pharmacy.

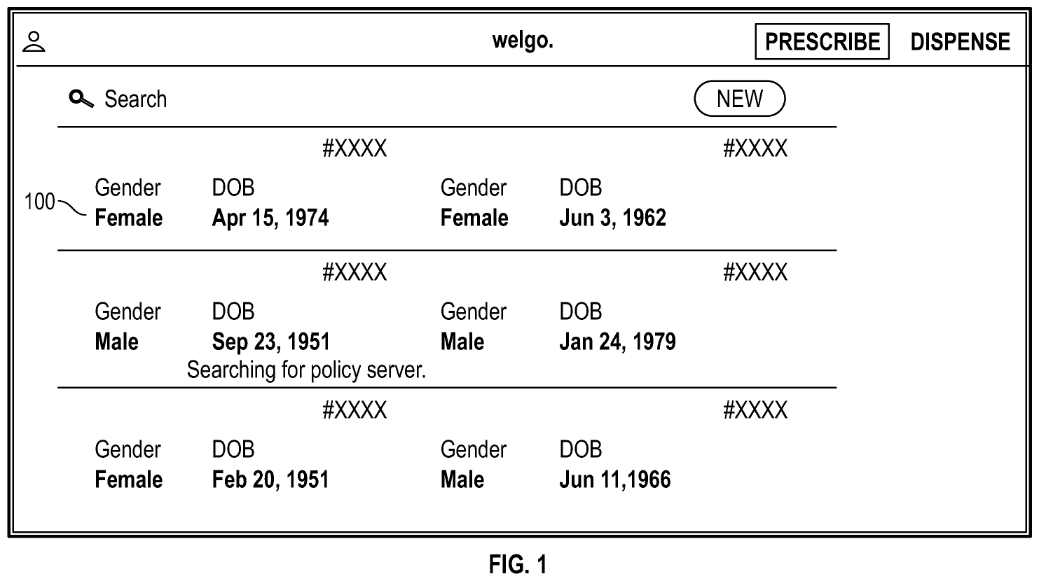

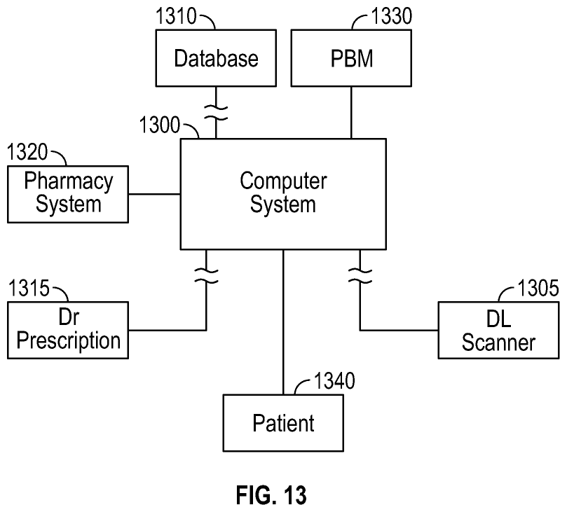

The hardware is shown in FIG. 13, operating according to the diagrams and flow diagrams of FIGS. 1-12. A computer system 1300, which can be located in one or more distributed locations, receives and provides all of the information. This computer system can be a cloud computing system, which can be communicated with over the Internet or other network.

An embodiment uses a driver's license scanner system 1305 to allow doctors or technicians to scan the patient's driver's license. That information from the driver's license including name, date of birth, gender, zip and sex of the patients is received into a file record 100, as shown in FIG. 1. The record 100 is used to look up the patient's PBM (pharmacy benefit manager) information from a database 1310.

In this way, that information does not need to be entered manually, e.g., from the patient's pharmacy card. This also avoids data entry errors.

As explained herein, the computer system also communicates with the doctor's prescription system 1315, the pharmacy system 1320, the PBM 1330 to approve prescriptions, and the patient 1340 to approve the specific medication which has been selected. Each of these different connections to each of these different parties may have their own user interface and display. For example, the patient may communicate with the system using their phone, while the pharmacy system and doctor systems may use existing medical computing systems.

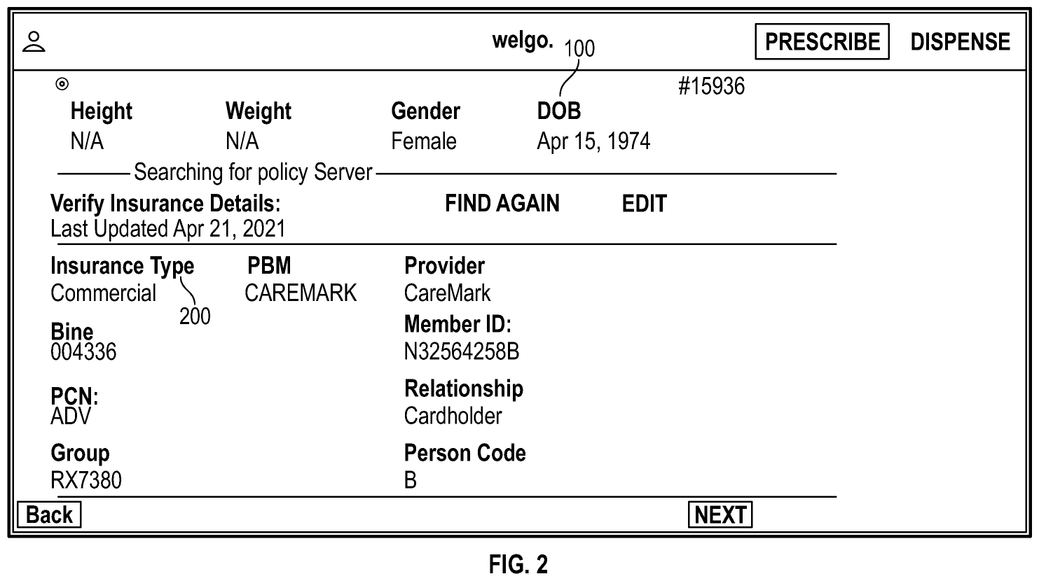

After the scanned information is used to look up the PBM information from the database 1310, FIG. 2 shows the PBM-specific information being sent to the PBM 1330. A return verification is received as 200 in FIG. 2. This begins the PBM claim.

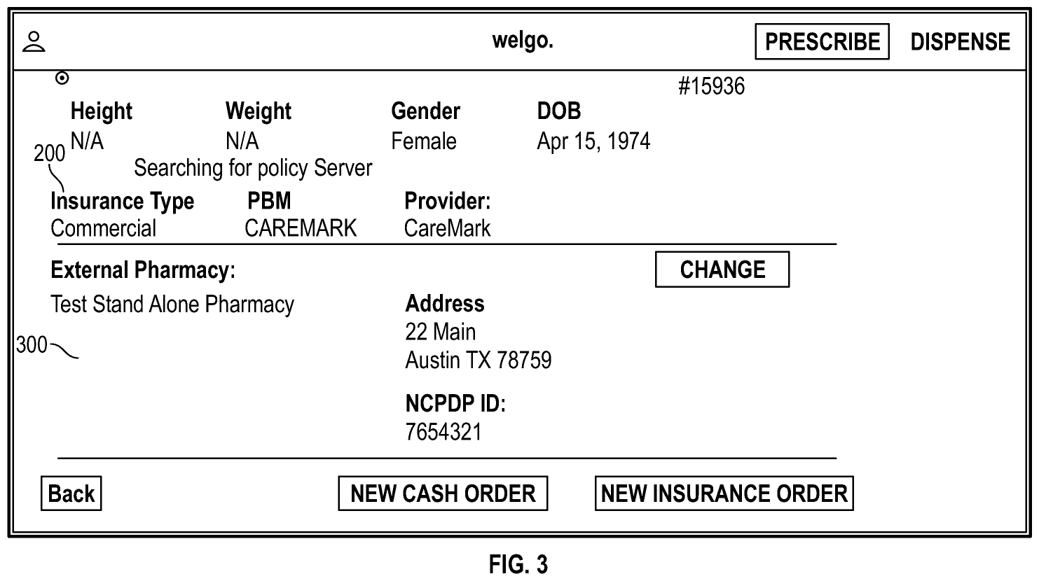

FIG. 3 shows the start of the PBM claim, using PBM information 200. An external pharmacy 300 is selected.

A portal to the doctor prescription system 1315 is also provided, which is the prescription system the doctors use to enter E prescriptions into the system. The computer system outputs the prescriptions to the pharmacy system 1320. The E prescriptions are submitted to the pharmacy 1310 using NCPDP standards and the response of the PBM is received. This response has a code that changes the nature of the information that needs to be submitted to the PBM so that the claim can be approved and processed. This coding process, for example, can vary the prescription as described herein, for example it can lower the amount of the medications that will be submitted to the PBM to allow that medication to be approved. Any PBM reject can be responded to in other ways to get the prescription approved, e.g, by changing the actual prescription or by automatically sending information to the PBM to support a diagnosis.

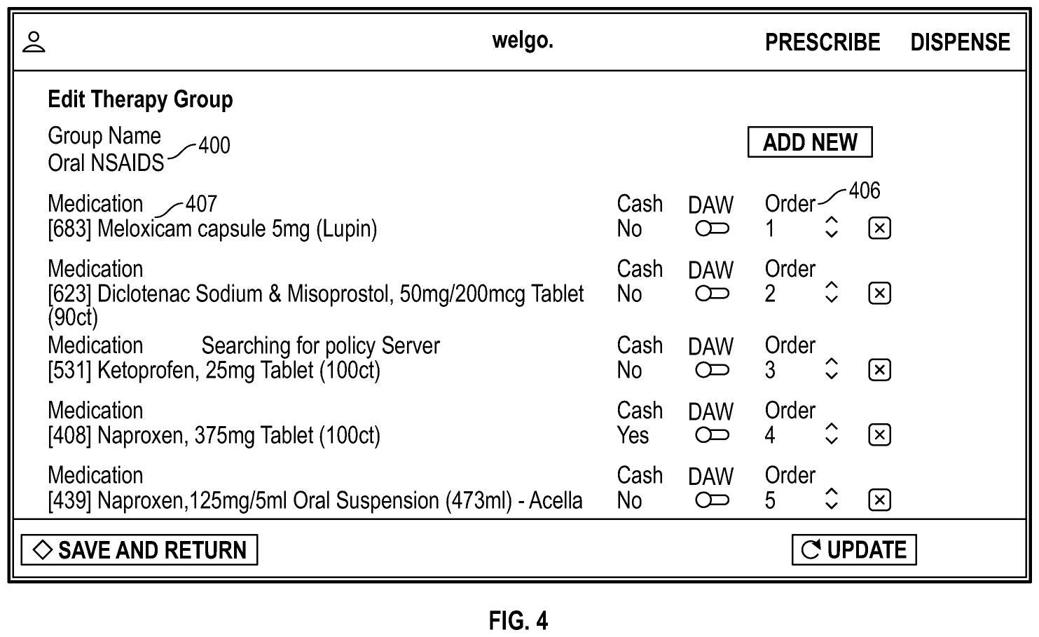

In an embodiment, the database 1310 stores, for each kind or class of medication, a hierarchy of medications and dosages that are likely to be allowed by the PBM, as shown in FIG. 4. This information about the hierarchy is displayed to the E prescriber. The hierarchy shows at the top the medications that are most likely to be approved. FIG. 4 shows the therapy group of NSAIDs arranged into a hierarchy, with an order of likelihood of prescription 406, for each medication 407. Within that group, the different drugs that can be used and prescribed are specified by order, shown in the right side as orders 1, 2, 3, 4, 5. This hierarchy can then be used to automatically determine which medications are most likely to be approved. FIG. 4 illustrates how medications are selected, with a priority extending from most likely to be covered to not covered, for individual doctor settings.

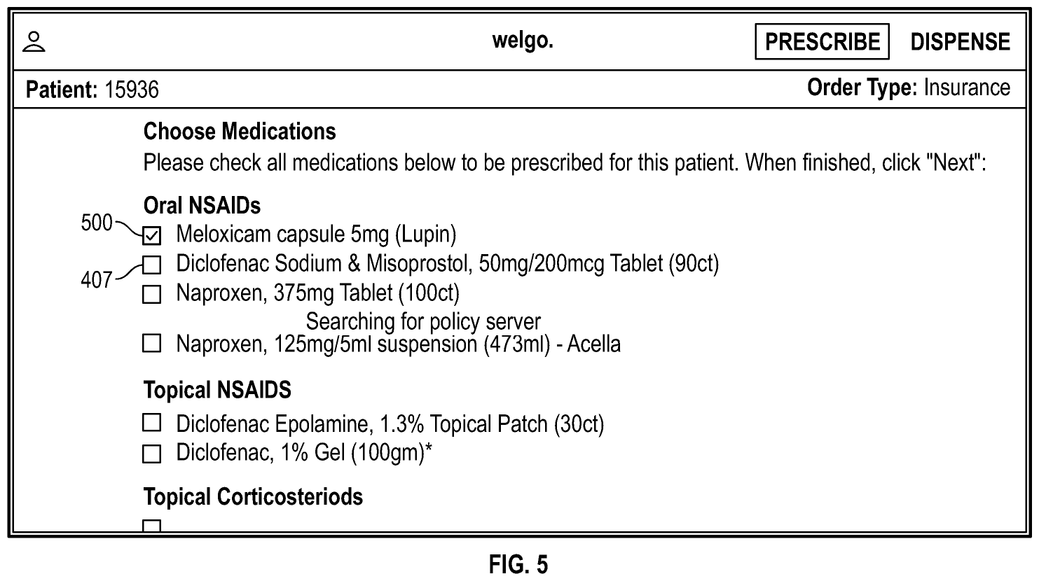

Based on this hierarchy, the eRx can be selected in FIG. 5. For the group, here oral NSAIDs, the different medications 407 are shown in order, to enable selecting the medication which is most likely to be approved for the specific patient.

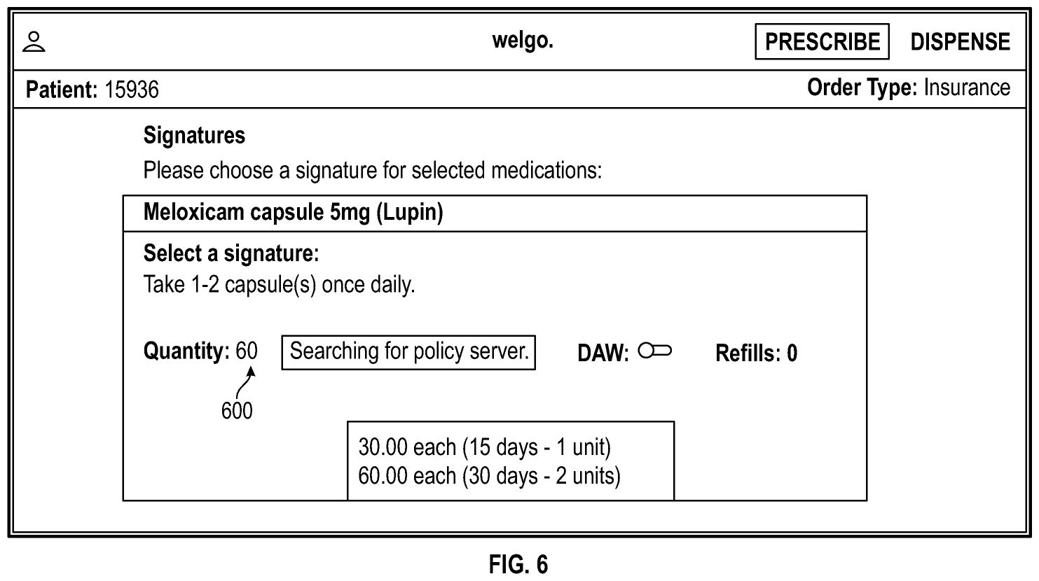

Once the medication is selected in FIG. 5, here medication 500 has been selected, FIG. 6 shows how this medication quantity being automatically set as quantity which will be approved by the PBM. This medication quantity is also stored in the database 1310. This can allow setting the number of days of supply of medication that the PBM will accept, and automatically setting the number of pills to be dispensed for that number of days. This can save tech time and avoid billing errors.

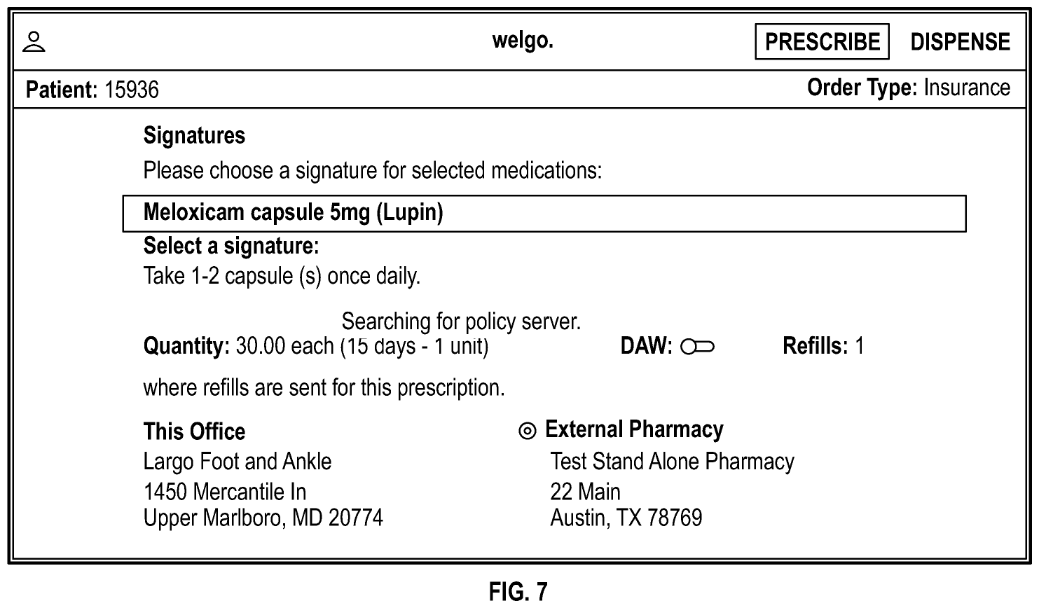

FIG. 7 illustrates the operation if refills are selected. The system can allow the original prescription to be sent to a first pharmacy, and the prescription to be sent to another pharmacy for example a mail order pharmacy for the remaining refills. This allows the user to obtain their first dose quickly from a local pharmacy, but allows the refills to be sent to another pharmacy.

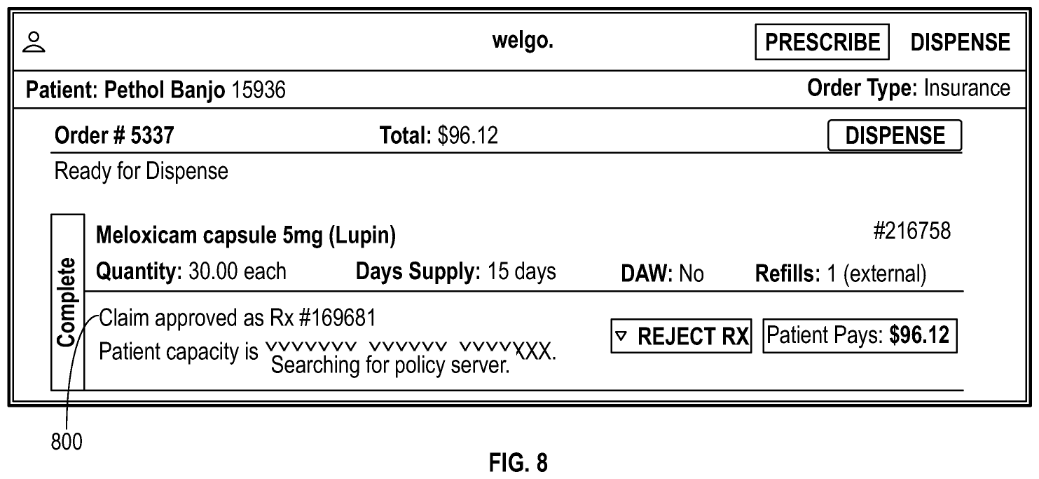

FIG. 8 illustrates the claim being submitted to the PBM and approved as 800.

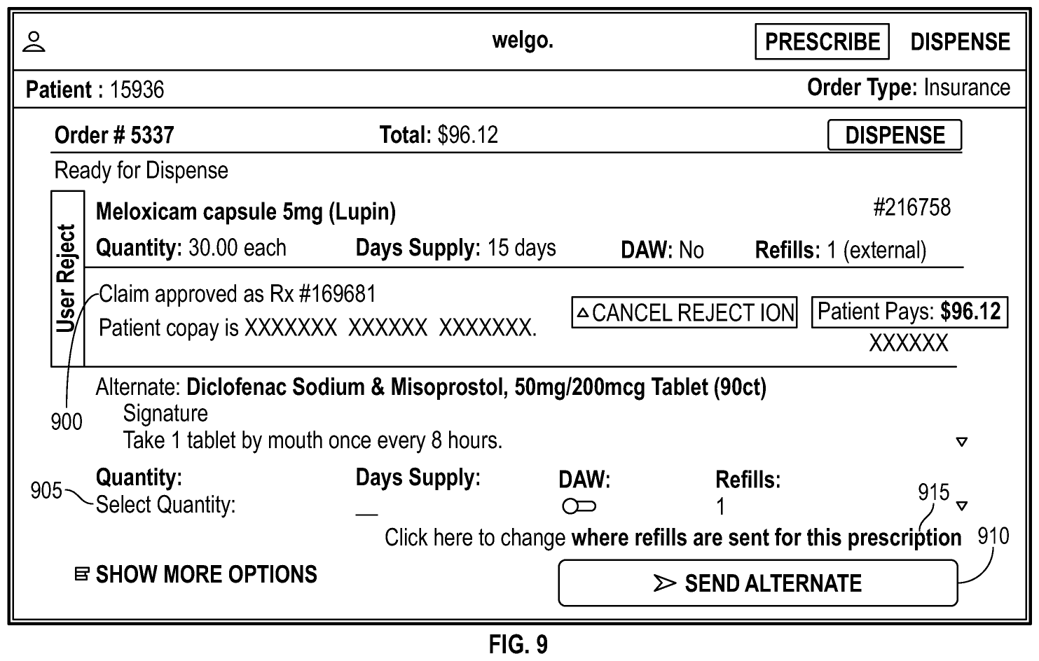

Once this claim is approved, it can be sent to the user, as 900 in FIG. 9, showing that the claim has been approved. The patient has the choice to accept or reject the specific medication. The patient, for example, can reject this, based on selection of an alternate medication shown as 905. If the user wants to select an alternate medication, they can request this alternate at 910, where it is sent to the PBM for approval. The user can also change where the medication refills are sent, to a different pharmacy than the original prescription fill, at 915.

The alternative medication can be the next medication in line from the hierarchy, or can be some other medication.

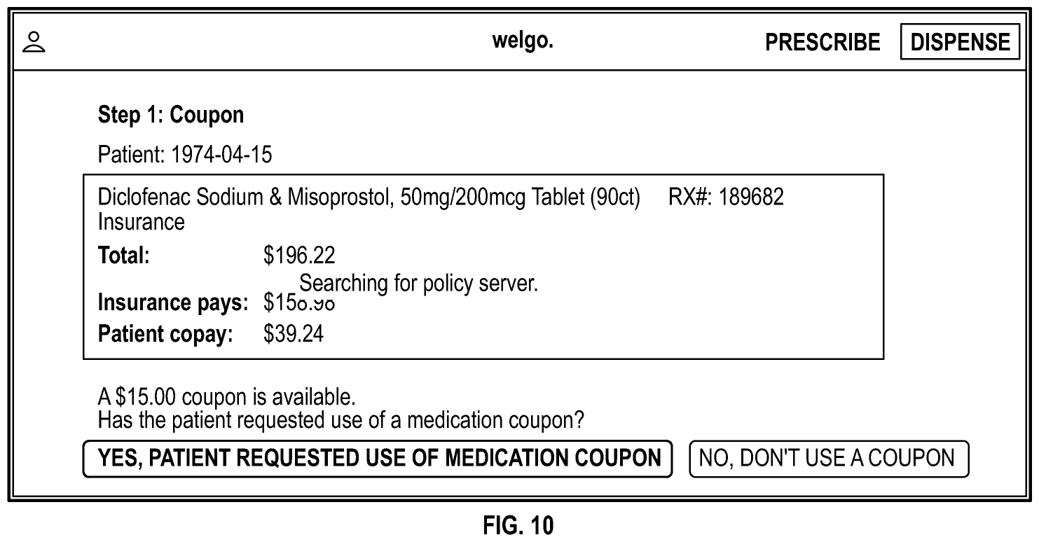

FIG. 10 illustrates how the medication is approved and a coupon is either applied, or not applied.

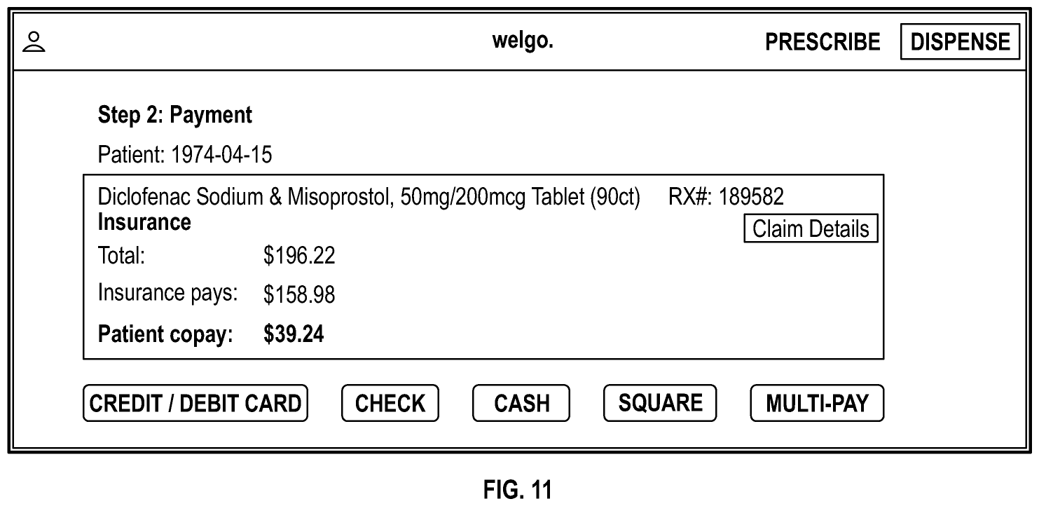

FIG. 11 illustrates the patient's co-pay being collected.

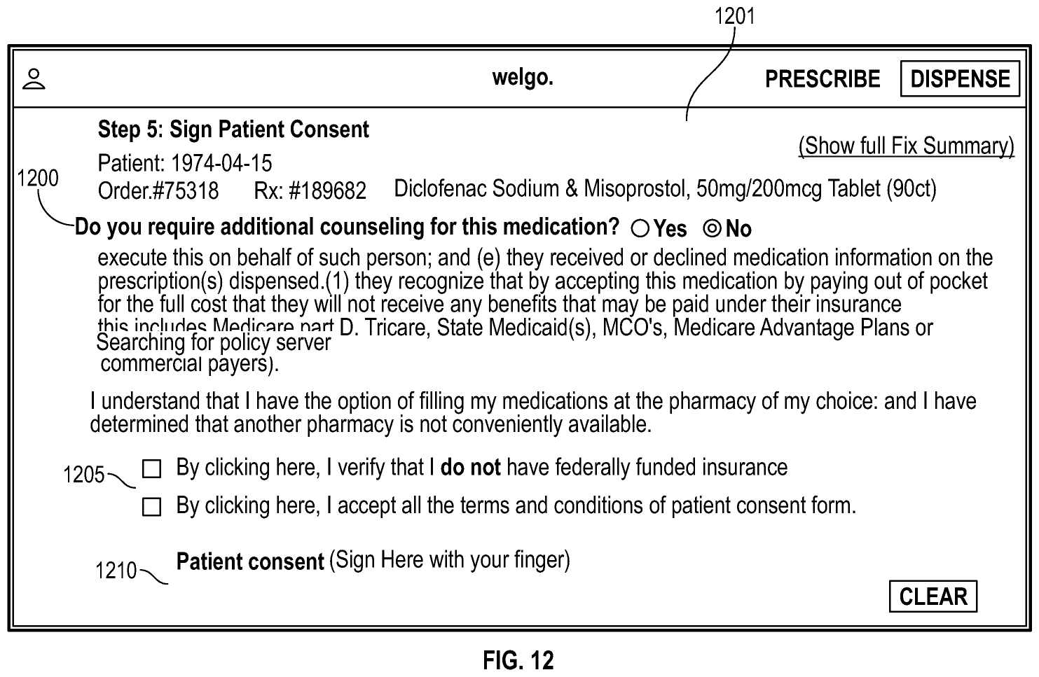

FIG. 12 shows the patient consent form 1201, initially requesting from the user whether they require additional counseling for the medication. The system also obtains verification information 1205 from the user, and obtains the patient's consent at 1210. This includes the patient allowing the patient to waive counseling for the medication.

Another feature, according to an embodiment, is use of the system as an inventory management system. According to an embodiment, the system is used to dispense medications at the actual clinic office that prescribes them. The inventory management system uses an order form that allows physicians to order initial medications from a wholesaler. The order form also has has a min/max feature which allows medications to be automatically shipped to the clinic once the levels fall below the min.

Another embodiment allows the clinics to pick from a number of wholesalers/manufactures, in which each wholesaler/manufacture offers their best price for a specific medication to the clinic. In an embodiment, the wholesalers/manufactures would pay a fee to have access to our platform to sell these medications for a percentage type fee—this would allow wholesalers/manufactures a new way to get their medications moved at a low cost for doing so.

Another embodiment provides a direct link with our software to the wholesalers/manufactures ERP system which would notify them when a medication is needed to ship to the clinic.

According to another embodiment, there is a web and app based data stream to manufactures sales teams which shows, in real time, what medications that physician is writing for a patients. This can provide real-time data to the sales reps and others about what is being prescribed. This compares with other systems which can only give information about past data, such as 60 days in the past. This allows the sales reps, for example, to get real-time data about what impact they had on the doctor.

Another embodiment allows the software to pull data from the electronic medical records and automatically submit the data for prior authorization at the point of care—rather than a retail pharmacy. This would allow pharma to decease the abandonment rates of their medications that occur at the retail pharmacy because lack of communication from the physician's office.

The system produces one or more of the following advantages in addition to the above.

Remove or reduce call backs from retail pharmacies to change Rx

Easily allow doctors to supplement medications for non-covered medications.

Ease of use using patient's driver's license or other ID to obtain and submit PBM information.

Remove pharmaceutical coupons from being used for Federally funded patients.

Remove the knowledge and legwork of a pharmacy tech from resubmitting information for pharmacy rejects codes via the disclosed code trees.

Combined e-prescribing system with a pharmacy claims adjudication system into a closed system to reduce time, cost and increase compliance.

The system can also include other information in the database that can also carry out other things that are normally done manually by a pharmacy tech. By allowing the system to automatically modify the prescription, this may minimize the need for a doctor's office to hire a certified pharmacy tech instead using the machine intelligence to carry out this kind of information.

The system can also filter the claim so that they cannot be submitted to excluded PBMs. This is important because, for example, federal law prohibits the use of coupons for federal patients. By filtering in this way, this helps ensure compliance with state and federal laws.

Although only a few embodiments have been disclosed in detail above, other embodiments are possible and the inventors intend these to be encompassed within this specification.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An electronic prescription system, comprising:
a computer, receiving information from a patient indicative of the patient's identity, and receiving information from a medical professional, indicative of a type of medication to be dispensed to the patient,
the computer looking up insurance information about the patient based on the information received from the patient;
the computer including a database, which stores information indicating, for the type of medication to be dispensed to the patient, a hierarchy of different medications which are most likely to be approved by an insurance represented by the insurance information,
the database also storing information about quantities of the medications which are likely to be approved;
the computer causing the hierarchy to be displayed to the medical professional and to enable the medical professional to select a medication to be dispensed, and
automatically using the stored information about the quantities of the medication to select a quantity of the medications to be dispensed.

2. The system as in claim 1, further comprising an identification scanner, which automatically scans a user's identification, to obtain the information about the patient's identity.

3. The system as in claim 2, wherein the identification scanner is a driver's license scanner, which automatically obtains name of the patient, and date of birth of the patient, and uses the name and date of birth to look up insurance information from the database.

4. The system as in claim 1, wherein the hierarchy includes a number of different medications for a therapy group.

5. The system as in claim 1, wherein the computer displays the medication to the patient and also displays an alternative medication to the patient, and enables the patient to request the alternative medication in place of the medication that was selected.

6. The system as in claim 1, wherein the computer system also allows prescribing refills.

7. The system as in claim 6, wherein the system allows an initial prescription to be filled by a first pharmacy, and refills to be filled by a second pharmacy different than the first pharmacy.

8. The system as in claim 1, further comprising the computer displaying information to a user information requesting the user to accept terms and conditions and determining if the user wants counseling for the medication.
